(19) United States
(12) Patent Application Publication
(10) Pub. No.: US 2022/0325421 A1
(43) Pub. Date: Oct. 13, 2022

(54) METHOD FOR INHIBITING REDUCTION IN CONCENTRATION OF OXIDIZING AGENT IN SULFURIC ACID SOLUTION CONTAINING PERSULFURIC ACID COMPONENT

(71) Applicant: KURITA WATER INDUSTRIES LTD., Tokyo (JP)

(72) Inventors: Hajime ISERI, Tokyo (JP); Haruyoshi YAMAKAWA, Tokyo (JP); Yuzuki YAMAMOTO, Tokyo (JP)

(21) Appl. No.: 17/639,158

(22) PCT Filed: Feb. 25, 2020
(86) PCT No.: PCT/JP2020/007427

§ 371 Date: Feb. 28, 2022

(30) Foreign Application Priority Data
Sep. 13, 2019 (JP) 2019-166956

(51) Int. Cl. C23G 1/06; C01B 15/08; C07D 249/18

(52) U.S. Cl. CPC C23G 1/061; C01B 15/08; C07D 249/18

(57) ABSTRACT

A heterocyclic compound having a structure in which a benzene ring and a nitrogen-containing heterocycle are condensed is added to a sulfuric acid solution that contains a persulfuric acid component such as peroxomonosulfuric acid, peroxomonosulfate, peroxodisulfuric acid, or peroxodisulfate as an oxidant and in which impurities that promote the reduction in the oxidant concentration are present. Here, the heterocyclic compound having a structure in which a benzene ring and a nitrogen-containing heterocycle are condensed is preferably a benzotriazole-based compound. The impurities that promote a reduction in the oxidant concentration are suitably one or more selected from copper ions, iron ions, nitrate ions, and nitrite ions. The method of the present invention of inhibiting a reduction in the oxidant concentration in a sulfuric acid solution containing a persulfuric acid component can inhibit the reduction in the oxidant concentration to a minimum even when impurities that cause a reduction in the oxidant concentration are mixed.

### TECHNICAL FIELD

The present invention relates to a method of inhibiting a reduction in the oxidant concentration in a sulfuric acid solution that contains a persulfuric acid component and is used for surface washing and surface modification treatment for various materials such as metal, silicon, glass, and plastic.

### BACKGROUND ART

A sulfuric acid solution containing a persulfuric acid component such as peroxomonosulfuric acid, peroxomonosulfate, peroxodisulfuric acid, or peroxodisulfate has extremely strong oxidizing properties. Utilizing such strong oxidizing properties, the sulfuric acid solution containing a persulfuric acid component is used for washing and modifying the surfaces of various materials such as metal, silicon, glass, and plastic.

For example, Patent Document 1 describes a method of producing a porous film. This method has a step of forming fine pores in an aluminum film through circulating a sulfuric acid-containing treatment liquid for a metal material in an electrolytic cell to generate a persulfuric acid and anodizing the aluminum film in the treatment liquid having a redox potential of +1.5 to +3.5 V. Patent Document 2 describes a sulfuric acid recycle type washing system in which when contaminants and the like adhering to silicon wafers or the like are washed for removal by using a persulfuric acid solution having a high removal effect, the persulfuric acid is recovered and used for washing while repeatedly using a sulfuric acid solution. Patent Document 3 describes a plastic surface treatment method as a pretreatment for plating. This method includes preparing a solution in which a persulfate is dissolved with a sulfuric acid concentration of 50 to 92 wt % and a persulfuric acid concentration of 3 to 20 g/L, setting the temperature of the solution in which the persulfate is dissolved to 80° C. to 140° C., and treating a plastic material with the solution.

### PRIOR ART DOCUMENTS

### Patent Documents

### [Patent Document 1] JP2016-145381A

### [Patent Document 2] JP2006-278689A

### [Patent Document 3] JP6288213B

### SUMMARY OF THE INVENTION

### Problems to be Solved by the Invention

As described in Patent Documents 1 to 3, a sulfuric acid solution containing a persulfuric acid component can be widely used for surface washing and surface modification for various materials, but the oxidant concentration in the solution is reduced in a short time and the oxidative activity may be lost. As a result of investigating the cause of this, the present inventors have found that the oxidant concentration is reduced in a short time when impurities are mixed in the sulfuric acid solution containing a persulfuric acid component. It has also been found that the reduction in the oxidant concentration is remarkable when the impurities are metal ions represented by copper ions and iron ions, nitrate ions, nitrite ions, or other similar ions.

In a situation in which the reduction in the oxidant concentration is remarkable due to the presence of such impurities, it is necessary to increase the supply rate of the persulfuric component in order to maintain the oxidant concentration in the solution at a concentration required for the surface treatment. For example, when supplying a persulfuric acid component generated by electrolyzing a sulfuric acid-containing liquid, it is necessary to increase the size of the electrolytic apparatus. When using a persulfuric acid component generated by a method of adding persulfate to sulfuric acid or a method of adding hydrogen peroxide to sulfuric acid, it is necessary to increase the amount of necessary components to be used, such as sulfuric acid, persulfate, and hydrogen peroxide. Thus, the mixing of impurities that cause a reduction in the oxidant concentration involves a problem in that not only the surface treatment effect of the sulfuric acid solution containing a persulfuric acid component is lowered, but also the treatment cost is increased.

The present invention has been made in view of the above problem, and an object of the present invention is to provide a method of inhibiting a reduction in the oxidant concentration in a sulfuric acid solution containing a persulfuric acid component with which it is possible to inhibit the reduction in the oxidant concentration to a minimum and allow the effect of a desired surface treatment to be exerted to a maximum even when an impurity that causes a reduction in the oxidant concentration is mixed.

### Means for Solving the Problems

To achieve the above object, the present invention provides a method of inhibiting a reduction in an oxidant concentration in a sulfuric acid solution that contains a persulfuric acid component as an oxidant and in which an impurity that promotes the reduction in the oxidant concentration is present, the method comprising adding a heterocyclic compound to the sulfuric acid solution in which the impurity that promotes the reduction in the oxidant concentration is present, the heterocyclic compound having a structure in which a benzene ring and a nitrogen-containing heterocycle are condensed (Invention 1).

According to the invention (Invention 1), even when an impurity that causes a reduction in the oxidant concentration is mixed in the sulfuric acid solution containing a persulfuric acid component, the reduction in the oxidant concentration in the sulfuric acid solution can be effectively inhibited and it is possible to allow the effect of surface treatment for various materials to be exerted to a maximum. Although the reason why such an effect is obtained is not necessarily clear, it is considered that the heterocyclic compound having a structure in which a benzene ring and a nitrogen-containing heterocycle are condensed acts on the impurity, which promotes a reduction in the oxidant concentration, and inhibits the reaction between the impurity and the oxidant.

In the above invention (Invention 1), the persulfuric acid component may be preferably at least one selected from peroxomonosulfuric acid, peroxomonosulfate, peroxodisulfuric acid, and peroxodisulfate (Invention 2).

According to the invention (Invention 2), these persulfate components each serve as an oxidant, but the presence of an impurity that promotes a reduction in the oxidant concentration causes a rapid reduction in the oxidant concentration, and this can therefore be inhibited by adding a heterocyclic compound having a structure in which a benzene ring and a nitrogen-containing heterocycle are condensed.

In the above invention (Invention 1, 2), the heterocyclic compound having a structure in which a benzene ring and a nitrogen-containing heterocycle are condensed may be preferably a benzotriazole-based compound (Invention 3).

According to the invention (Invention 3), the benzotriazole-based compound can suitably inhibit the reaction between the impurity that promotes the reduction in the oxidant concentration and the oxidant, and the reduction in the oxidant concentration in the sulfuric acid solution can be effectively inhibited.

In the above invention (Invention 1 to 3), the impurity that promotes the reduction in the oxidant concentration may preferably one or more selected from copper ion, iron ion, nitrate ion, and nitrite ion (Invention 4).

According to the invention (Invention 4), the copper ion, iron ion, nitrate ion, and nitrite ion are likely to cause a reduction in the oxidant concentration in the sulfuric acid solution containing a persulfuric acid component and are also likely to be industrially mixed as impurities, but it is possible to effectively inhibit the reduction in the oxidant concentration in the sulfuric acid solution due to their effects.

### Advantageous Effect of the Invention

According to the method of the present invention of inhibiting a reduction in the oxidant concentration in a sulfuric acid solution containing a persulfuric acid component, even when an impurity that causes a reduction in the oxidant concentration is mixed in the sulfuric acid solution containing a persulfuric acid component, the reduction in the oxidant concentration in the solution can be effectively inhibited and it is possible to allow the effect of surface treatment for various materials to be exerted to a maximum. Moreover, the oxidant concentration in the solution required for the surface treatment is maintained, and the supply rate of the persulfuric acid component can thereby be suppressed to the minimum necessary. These enable the treatment to be performed with a compact electrolytic apparatus when supplying a persulfuric acid component generated, for example, by electrolyzing a sulfuric acid-containing liquid. Furthermore, when using a persulfuric acid component generated by a method of adding persulfate to sulfuric acid or a method of adding hydrogen peroxide to sulfuric acid, it is possible to reduce the amount of each component to be used, such as sulfuric acid, persulfate, or hydrogen peroxide.

### BRIEF DESCRIPTION OF DRAWINGS

FIG. 1 is a graph illustrating changes in the oxidant concentration when copper ions, iron ions, nitrate ions, and nitrite ions are added to a sulfuric acid solution containing a persulfuric acid component.

FIG. 2 is a graph illustrating changes in the oxidant concentration of Example 1 and Comparative Example 1.

### EMBODIMENTS FOR CARRYING OUT THE INVENTION

The method of the present invention of inhibiting a reduction in the oxidant concentration in a sulfuric acid solution containing a persulfuric acid component will be described in detail based on the following one or more embodiments.

### <Method of Inhibiting Reduction in Oxidant Concentration in Sulfuric Acid Solution Containing Persulfuric Acid Component>

### (Sulfuric Acid Solution Containing Persulfuric Acid Component)

In the present embodiment, the sulfuric acid solution containing a persulfuric acid component is not particularly limited, provided that it contains a persulfuric acid component and is a sulfuric acid solution. Examples of the persulfuric acid component include peroxomonosulfuric acid, peroxomonosulfate, peroxodisulfuric acid, and peroxodisulfate. These may each be appropriately selected and used alone, or two or more types may also be used in combination.

Examples for use as the sulfuric acid solution containing a persulfuric acid component include a sulfuric acid solution (electrolytic sulfuric acid solution) obtained by electrolyzing a sulfuric acid-containing liquid to generate a persulfuric acid component and a solution obtained by adding hydrogen peroxide to sulfuric acid to generate a persulfuric acid component. In particular, the present invention can be suitably applied to the electrolytic sulfuric acid solution.

When the sulfuric acid solution containing a persulfuric acid component is an electrolytic sulfuric acid solution, for example, the sulfuric acid concentration may be preferably 60 to 87 wt % and particularly preferably 70 to 83 wt %. The oxidant concentration in the initial state may be 2 g/L or more and particularly 3 to 20 g/L depending on the intended use. If the oxidant concentration is less than 2 g/L, the influence of the reduction in the oxidant concentration may not be so large, while if the oxidant concentration exceeds 20 g/L, the production itself may not be economical and the oxidizing ability may not be problematic even when the oxidant concentration is reduced to a certain degree.

(Impurities that Promote Reduction in Oxidant Concentration)

Impurities that promote the reduction in the concentration of an oxidant mixed in the sulfuric acid solution containing the persulfuric acid component as described above are not particularly limited, provided that the impurities react with and consume the oxidant component in the sulfuric acid solution containing a persulfuric acid component. Examples of such impurities include copper ions, iron ions, nitrate ions, and nitrite ions, which are likely to be mixed as impurities because they are often used industrially. Among these, when copper ions and iron ions, particularly copper ions, are mixed as impurities, the reduction in the oxidant concentration can be suitably inhibited.

The concentration of the impurities, which promote the reduction in the oxidant concentration as described above, in the sulfuric acid solution containing a persulfuric acid component may be about 5000 ppm or less and particularly about 3000 to 1 ppm. If the impurity concentration exceeds 5000 ppm, the amount of impurities is too large, and the effect of inhibiting the reduction in the oxidant concentration in the solution may not be sufficiently exhibited. The lower limit of the impurity concentration is not particularly limited, but if it is less than 1 ppm, the reduction in the oxidant concentration is not large and it is difficult to confirm the effect, which may not be preferred.

### (Oxidant Concentration Reduction Inhibitor)

In the present embodiment, a heterocyclic compound having a structure in which a benzene ring and a nitrogen-containing heterocycle are condensed is used as an oxidant concentration reduction inhibitor. Examples of the heterocyclic compound having a structure in which a benzene ring and a nitrogen-containing heterocycle are condensed include 1,2,3-benzotriazole, 5-methylbenzotriazole, 4-methylbenzotriazole, 5,6-dimethylbenzotriazole, benzoimidazole, and 5,6-dimethylbenzoimidazole, among which benzotriazole compounds typified by 1,2,3-benzotriazole and 5-methylbenzotriazole may be particularly suitable.

The addition concentration of the oxidant concentration reduction inhibitor may be 0.01 times or more and further preferably 0.05 times or more with respect to the molar concentration of impurities that cause a reduction in the concentration of an oxidant contained in the solution. The upper limit of the addition amount may be preferably 1 times or less and particularly preferably 0.5 times or less with respect to the molar concentration of impurities that cause a reduction in the oxidant concentration because if it is too large, the oxidant concentration reduction inhibitor may affect the effect of treatment such as surface treatment with a sulfuric acid solution containing a persulfuric acid component.

The method of adding the oxidant concentration reduction inhibitor as described above is not particularly limited, provided that it is possible to achieve a state in which the required concentration is dissolved in the sulfuric acid solution containing a persulfuric acid component. The oxidant concentration reduction inhibitor may be added and dissolved in a state of solid (powder) in the sulfuric acid solution containing a persulfuric acid component or may otherwise be added in a state of liquid by preliminarily dissolving the oxidant concentration reduction inhibitor in a solution. For example, when a sulfuric acid-containing liquid is electrolyzed to generate a persulfuric acid component, the heterocyclic compound may be added to the solution after electrolysis, or a sulfuric acid solution in which the heterocyclic compound is preliminarily dissolved may be electrolyzed. When the persulfuric acid component is generated by a method of adding persulfate to sulfuric acid or a method of adding hydrogen peroxide to sulfuric acid, the heterocyclic compound may be added to a sulfuric acid solution containing the generated persulfuric acid component, or a sulfuric acid solution containing a persulfuric acid component can be generated by using sulfuric acid, hydrogen peroxide, or the like to which the heterocyclic compound is preliminarily added.

The method of the present invention of inhibiting the reduction in the oxidant concentration in the sulfuric acid solution containing a persulfuric acid component has been described above, but the present invention is not limited to the above embodiment and various modifications can be carried out. For example, the sulfuric acid solution containing a persulfuric acid component may not have to be composed only of the persulfuric acid component and the sulfuric acid solution, and may contain other acids such as phosphoric acid and/or a chemical liquid component if they do not reduce the oxidant concentration.

### EXAMPLES

The present invention will be specifically described with reference to the following Examples and Comparative Examples. Note, however, that the present invention is not limited to these descriptions.

### Reference Example

A confirmation test was conducted to confirm the reduction in the oxidant concentration by using a test liquid generated by electrolyzing 78 wt % sulfuric acid (H₂SO₄) so that the oxidant concentration would be 7 to 8 g/L as S₂O₈. The oxidant concentration was measured by an iodine titration method. The iodine titration method refers to a method that includes adding KI to a small amount of a sampled test liquid to liberate I₂, titrating the I₂ with a Na₂S₂O₃ standard solution to obtain the amount of I₂, and obtaining the oxidant concentration from the amount of I₂.

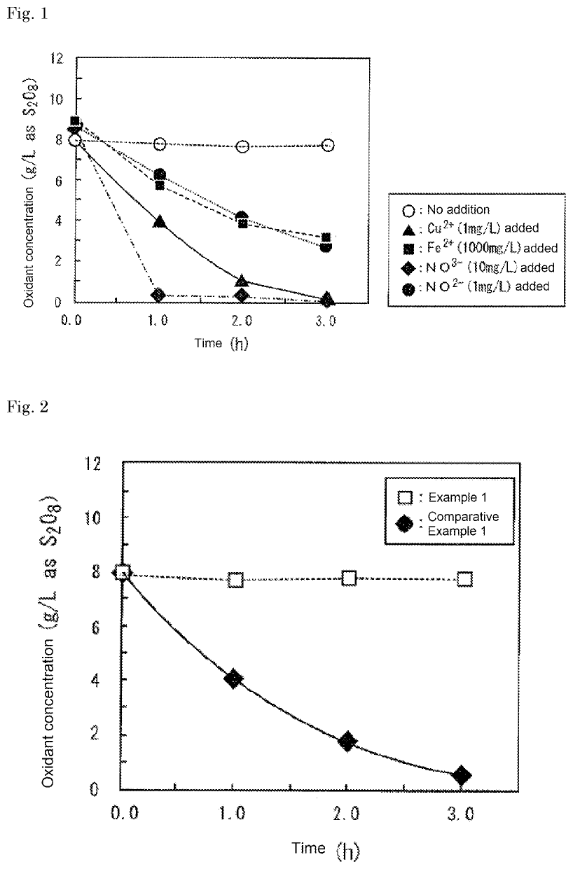

To 200 mL of the test liquid heated to 60° C., 1 mg/L of copper ions (Cu²⁺), 1000 mg/L of iron ions (Fe²⁺), 10 mg/L of nitrate ions (NO³⁻), or 1 mg/L of nitrite ions (NO²⁻) was added. Stirring was continued with a stirrer while keeping the temperature of the test liquid constant, and the oxidant concentration in the test liquid was measured every hour until the lapse of 3 hours. The results are illustrated in FIG. 1. In addition, for comparison, the results of measuring the oxidant concentration in the test liquid every hour until the lapse of 3 hours are also illustrated in FIG. 1 for the test liquid without addition of these ionic components.

As apparent from FIG. 1, it has been confirmed that the oxidant concentration in the test liquid is kept constant in the case of no addition, while when copper ions, iron ions, nitrate ions, or nitrite ions are added to the sulfuric acid solution containing the persulfuric acid component as an oxidant, the oxidant concentration in the test liquid is reduced in a short time.

### Examples 1 to 11 and Comparative Examples 1 to 6

To 200 mL of the test liquid heated to 60° C. and used in the above Reference Example, 1,2,3 benzotriazole, 5-methylbenzotriazole, benzoimidazole, 1-hydroxyethane-1,1-diphosphonic acid (HEDP), phosphoric acid, or ethylenediaminetetraacetic acid-tetrasodium (EDTA-4Na) was added and dissolved as an oxidant concentration reduction inhibitor as listed in Tables 1 and 2 so as to have a concentration listed in Tables 1, 2, and 3. Subsequently, copper ions, iron ions, or nitrate ions were added as impurity components causing a reduction in the oxidant concentration so as to have a concentration listed in Tables 1, 2, and 3. Stirring was continued with a stirrer while keeping the temperature of the test liquid constant, and the oxidant concentration in the test liquid was measured after the lapse of 3 hours. The results are listed in Tables 1, 2 and 3 together with the oxidant concentration reduction inhibition rate. In addition, for Example 1 and Comparative Example 1, the oxidant concentration in the test liquid was measured every hour until the lapse of 3 hours. The results are illustrated in FIG. 2.

In Tables 1 to 3, the oxidant concentration reduction inhibition rate is calculated in accordance with the following equation.

Oxidant concentration reduction inhibition rate (%)=(R₁−R_{M})/(100−R_{M})×100

(In the equation, R_{M} represents an oxidant residual rate under a condition in which the oxidant concentration reduction factor is added and the inhibitor is not added, and R₁ represents an oxidant residual rate under a condition in which the oxidant concentration reduction factor of the same type as in the condition for obtaining R_{M} is added and the inhibitor is added.)

TABLE 1

| | | Example | Example | Example | Example | Example | Example |
|---|---|---|---|---|---|---|---|
| | | 1 | 2 | 3 | 4 | 5 | 6 |
| Oxidant | Copper ions (mg/L as Cu²⁺) | 1 | 1000 | 1000 | 1000 | 1000 | 1 |
| concentration | Iron ions (mg/L as Fe²⁺) | 0 | 0 | 0 | 0 | 0 | 0 |
| reduction | Nitrate ions (mg/L as NO³⁻) | 0 | 0 | 0 | 0 | 0 | 0 |
| factor | Nitrite ions (mg/L as NO²⁻) | 0 | 0 | 0 | 0 | 0 | 0 |
| Inhibitor | 1,2,3 benzotriazole (mg/L) | 1000 | 100 | 500 | 1000 | 0 | 0 |
| | 5-methylbenzotriazole (mg/L) | 0 | 0 | 0 | 0 | 1000 | 0 |
| | Benzoimidazole (mg/L) | 0 | 0 | 0 | 0 | 0 | 1000 |

| | | | | | | |
|---|---|---|---|---|---|---|
| Oxidant concentration reduction inhibition | 97.5 | 92.2 | 98.6 | 100.0 | 88.8 | 49.4 |
| rate (%) | | | | | | |

TABLE 2

| | | Example | Example | Example | Example | Example |
|---|---|---|---|---|---|---|
| | | 7 | 8 | 9 | 10 | 11 |
| Oxidant | Copper ions (mg/L as Cu²⁺) | 0 | 0 | 0 | 0 | 0 |
| concentration | Iron ions (mg/L as Fe²⁺) | 100 | 1000 | 0 | 0 | 0 |
| reduction | Nitrate ions (mg/L as NO³⁻) | 0 | 0 | 1 | 10 | 0 |
| factor | Nitrite ions (mg/L as NO²⁻) | 0 | 0 | 0 | 0 | 1 |
| Inhibitor | 1,2,3 benzotriazole (mg/L) | 1000 | 1000 | 1000 | 1000 | 1000 |
| | 5-methylbenzotriazole (mg/L) | 0 | 0 | 0 | 0 | 0 |
| | Benzoimidazole (mg/L) | 0 | 0 | 0 | 0 | 0 |

| | | | | | |
|---|---|---|---|---|---|
| Oxidant concentration reduction inhibition | 83.3 | 52.5 | 69.1 | 30.3 | 77.3 |
| rate (%) | | | | | |

TABLE 3

| | | Comparative | Comparative | Comparative | Comparative | Comparative | Comparative |
|---|---|---|---|---|---|---|---|
| | | Example 1 | Example 2 | Example 3 | Example 4 | Example 5 | Example 6 |
| Oxidant | Copper ions (mg/L as Cu²⁺) | 1 | 1 | 1 | 0 | 0 | 0 |
| concentration | Iron ions (mg/L as Fe²⁺) | 0 | 0 | 0 | 100 | 100 | 0 |
| reduction | Nitrate ions (mg/L as NO³⁻) | 0 | 0 | 0 | 0 | 0 | 1 |
| factor | Nitrite ions (mg/L as NO²⁻) | 0 | 0 | 0 | 0 | 0 | 0 |
| Inhibitor | HEDP (mg/L as PO₄) | 1000 | 0 | 0 | 1000 | 0 | 0 |
| | Phosphoric acid (mg/L as PO₄) | 0 | 1000 | 0 | 0 | 0 | 1000 |
| | EDTA-4Na (mg/L) | 0 | 0 | 1000 | 0 | 1000 | 0 |

| | | | | | | |
|---|---|---|---|---|---|---|
| Oxidant concentration reduction inhibition | 4.9 | −19.8 | 0.0 | 15.8 | −1145.5 | −10.0 |
| rate (%) | | | | | | |

As apparent from Tables 1 to 3, in Examples 1 to 11 in which the benzoazole-based inhibitor was added to the test liquid to which copper ions, iron ions, nitrate ions, or nitrite ions were added as the oxidant concentration reduction factor, the effect of inhibiting the reduction in the oxidant concentration was recognized. In particular, the reduction inhibition rate in Examples 1 to 5 in which 1,2,3 benzotriazole or 5-methylbenzotriazole was added as an inhibitor to the test liquid containing copper ions was high. On the other hand, in Comparative Examples 1 to 6 in which HEDP, phosphoric acid, or EDTA-4Na was added to the test liquid to which copper ions, iron ions, nitrate ions, or nitrite ions were added as the oxidant concentration reduction factor, the effect of inhibiting the reduction in the oxidant concentration was low, or the reduction was rather promoted.

Furthermore, as apparent from FIG. 2, it has been confirmed that, in Example 1, the oxidant concentration exhibits an approximately constant value for 3 hours, and the reduction in the oxidant concentration due to the presence of copper ions can be inhibited, whereas in Comparative Example 1, the oxidant concentration is reduced significantly in a short time.

1. A method of inhibiting a reduction in an oxidant concentration in a sulfuric acid solution that contains a persulfuric acid component as an oxidant and in which an impurity that promotes the reduction in the oxidant concentration is present, the method comprising
adding a heterocyclic compound to the sulfuric acid solution in which the impurity that promotes the reduction in the oxidant concentration is present, the heterocyclic compound having a structure in which a benzene ring and a nitrogen-containing heterocycle are condensed.

2. The method of inhibiting a reduction in an oxidant concentration in a sulfuric acid solution that contains a persulfuric acid component according to claim 1, wherein the persulfuric acid component is at least one selected from peroxomonosulfuric acid, peroxomonosulfate, peroxodisulfuric acid, and peroxodisulfate.

3. The method of inhibiting a reduction in an oxidant concentration in a sulfuric acid solution that contains a persulfuric acid component according to claim 1, wherein the heterocyclic compound having a structure in which a benzene ring and a nitrogen-containing heterocycle are condensed is a benzotriazole-based compound.

4. The method of inhibiting a reduction in an oxidant concentration in a sulfuric acid solution that contains a persulfuric acid component according to claim 1, wherein the impurity that promotes the reduction in the oxidant concentration is one or more selected from copper ion, iron ion, nitrate ion, and nitrite ion.
